(19) United States
(12) Patent Application Publication
(10) Pub. No.: US 2012/0101563 A1
(43) Pub. Date: Apr. 26, 2012

(54) DELIVERY SYSTEM FOR BRANCHED STENT GRAFT

(76) Inventors: Qing Zhu, Shanghai (CN); Qiyi Luo, Shanghai (CN); Dingguo Huang, Shanghai (CN); Yanbin Gao, Shanghai (CN); Zaiping Jing, Shanghai (CN); Qingsheng Lu, Shanghai (CN)

(21) Appl. No.: 13/322,256

(22) PCT Filed: Apr. 28, 2010
(86) PCT No.: PCT/CN2010/072280

§ 371 Date: Jan. 12, 2012

(30) Foreign Application Priority Data
May 26, 2009 (CN) 200910052079.1

(51) Int. Cl. A61F 2/84

(52) U.S. Cl. 623/1.12

(57) ABSTRACT

A delivery system for a branched stent graft comprises a double-layer sheath (3, 13) containing the stent graft and a towing and releasing mechanism which tows and releases the stent graft from the double-layer sheath (3, 13). After being bound, a stent main body (8) and a stent branch (11) of the stent graft are movably and entirely positioned within the double-layer sheath (3, 13). The stent main body (8) is connected with a control guiding assembly and is towed and released by the control guiding assembly. The stent branch (11) is towed by a branch guiding assembly and is connected with the control guiding assembly, so as to be released by the control guiding assembly.

The present application claims a priority of the Chinese patent application with the application No. “200910052079.1”, entitled “Delivery System for Branched Stent Graft and Delivery Method thereof”, which was submitted with the Chinese Patent Office on May 26, 2009. All the contents of this Chinese patent application are incorporated into the present application by reference.

### TECHNICAL FIELD

The present invention relates to a medical device, in particular a delivery system which delivers a stent graft in vivo for treating arterial lesions, and especially a delivery system for a branched stent graft with branched delivery and a delivering method thereof

### BACKGROUND ART

The endovascular graft exclusion (EVGE) is a new technology rising in popularity in the 1990s. When compared with traditional treatment methods, the employment of the interventional technique has the advantages of smaller incisions, fewer complications, low mortality, etc.

However, at present, the EVGE is mainly applicable to lesions not involving a branch artery, but quite a part of clinical aortic expansion diseases involve the branch artery. It is very complicated to treat the branch artery where the lesions occur using traditional prosthetic vessel replacement, which involves larger incisions and a higher mortality. Thus, it is more essential to employ a minimally invasive method to perform the treatment. Unfortunately, although the prosthetic vessel stent graft and the technique for delivering it have been developed well, techniques relating to the branched stent graft are still quite few. The techniques for delivering the branched stent graft have become a problem to be urgently solved in the technical field of minimally invasive treatment.

### SUMMARY OF THE INVENTION

The object of the present invention is to mainly solve a problem of delivering a transplant to the location of the lesion, especially the location of the lesion of the branch artery in a minimally invasive operation concerning the aorta, especially the thoracic aorta and its branch artery.

To achieve the above object, the present invention provides a delivery system for a branched stent graft, wherein after the bound branched stent graft is encapsulated in a complete or semi-complete manner using a graft sleeve made of a flexible polymer material, the branched stent graft is movably enclosed in an outer-layer sheath made of a polymer material, and after a stent branch and a stent main body of the stent graft are delivered to locations of lesions of an aorta and a branch blood vessel in a safe and fluent manner, the stent graft is accurately positioned and safely released.

Specifically, a delivery system for a branched stent graft according to the present invention comprises a double-layer sheath containing the stent graft and a towing and releasing mechanism which tows and releases the stent graft from the double-layer sheath. After being bound, a stent main body and a stent branch of the stent graft are movably and entirely positioned within the double-layer sheath, the stent main body is connected with the control guiding assembly and is towed and released by the control guiding assembly, and the stent branch is towed by a branch guiding assembly and is connected with the control guiding assembly, so that the stent branch is released by the control guiding assembly.

The double-layer sheath comprises an outer-layer sheath and an inner-layer graft sleeve. Preferably, after being movably encapsulated by the graft sleeve in a complete or semi-complete manner, the stent branch and the stent main body are positioned in the outer-layer sheath.

According to one aspect of the present invention, before being positioned in the double-layer sheath, the stent branch is enclosed in a branch sheath and is bound by the branch sheath, so that the stent branch and the stent main body are isolated from each other. In a preferred embodiment, the control guiding assembly comprises a control guide wire and a corresponding control mechanism, and a main body coil detachably binds the stent main body and is connected to the control guide wire; the branch guiding assembly comprises a branch guide wire and a corresponding control mechanism, the branch sheath is detachably connected to the control guide wire, and the branch guide wire connects and tows the branch sheath. In this case, an operator can accurately position the stent branch before releasing the stent graft, especially before releasing the stent branch. With respect to the lesion of the branch blood vessel, this kind of design is especially favorable.

It should be understood that the stent branch can be also bound in other manners, e.g., the stent branch can be bound by a structure similar to the main body coil and is connected to the control guide wire, whereby safely releasing the stent main body and the stent branch simultaneously or separately can be also achieved by the control guide wire.

In order to prevent the stent branch from being released unexpectedly or in advance during the delivering process, the present invention provides such a design where the branch sheath is detachably connected to the control guide wire via a branch safety buckle on the branch sheath, and the safe release of the stent branch is controlled by means of the control guide wire.

In addition, in the present invention, the graft sleeve is preferably made of a flexible polymer material. The flexible polymer material is, for example, PET (polyethylene terephthalate), PTFE (polytetrafluoroethylene), etc. The outer-layer sheath is preferably made of a polymer material. This polymer material is, for example, HDPE (high density polyethylene), Pebax (polyether acidamide), etc.

The present invention further provides a method for delivering a branched stent graft to inside of an artery using the above-described delivery system for a branched stent graft, comprising:

a) a towing step for towing the stent graft to inside of the artery by a control guiding assembly, and moving the stent graft out of the double-layer sheath;

b) a positioning step for respectively adjusting positions of the stent main body and the stent branch by the control guiding assembly and the branch guiding assembly according to a location of a lesion;

c) a releasing step for making the control guiding assembly loosen binding of the stent graft to thereby release the stent graft.

More specifically, in the step a), it is preferable that the inner-layer sheath encapsulating the stent graft is moved out of the outer-layer sheath, and then the stent graft is moved out of the inner-layer sheath. In this case, as mentioned above, since the inner-layer graft sleeve of the present invention employs a flexible polymer material, after the inner-layer graft sleeve is moved out of the outer-layer sheath, the graft sleeve can be delivered through an extremely tortuous artery, and the stent graft contained in the inner-layer graft sleeve can be further easily released.

Advantageously, a pre-releasing step b′) is further included between the steps b) and c), wherein in the step b′), the stent branch still remains in a state of being bound, and the stent branch is towed by the branch guide wire to make the stent branch be separated from the stent main body and enter a branch blood vessel. In this pre-released state, the operator can perform a positioning adjustment of the stent branch and the stent main body, especially a positioning adjustment of the stent branch, according to the actual condition of the operation, to achieve an accurate positioning of the whole of the stent graft. Subsequently, the step c) is carried out to release the whole of the stent graft.

Compared with the prior art, the delivery system for a branched stent graft and the method for delivering the same provided by the present invention can be applied to the EVGE especially involving the lesion of the branch artery more widely. This delivery system is simple in structure and convenient in use. The employment of the double-layer sheath technique can effectively ensure that the stent graft is delivered to the inside of the artery in a fluent and safe manner. The pre-releasing technique for the stent branch and the stent main body can accurately control the position of the stent graft, especially the position of the stent branch of the stent graft, according to the location of the lesion. More particularly, the present invention can achieve safely releasing the stent main body and the stent branch of the stent graft simultaneously or separately after positioning them.

### BRIEF DESCRIPTION OF THE DRAWINGS

By means of the detailed descriptions of the non-restrictive embodiments made in accordance with the following figures, the other features, the object and the advantages of the present invention will become more obvious:

FIG. 1 is a schematic diagram of an external structure of the delivery system for a branched stent graft according to the present invention;

FIG. 2 is a partial schematic diagram, which shows a part of an internal structure of the delivery system in FIG. 1, wherein the double-layer sheath has encapsulated the branched stent graft;

FIG. 3 is a schematic diagram of delivering the delivery system with a branched stent graft in FIG. 2 to the inside of the artery, wherein the stent graft has been moved out of the double-layer sheath;

FIG. 4 is a schematic diagram of the stent graft in FIG. 3 being in a pre-released state;

FIG. 5 is a schematic diagram of the stent graft in FIG. 4 being in a completely-released state; and

FIG. 6 is a schematic diagram of the double-layer sheath with a branched stent graft, which shows a connective mode of two layers of sheaths.

Explanations of Reference Signs

| | |
|---|---|
| 1 conical head | 2 branch guide wire |
| 3 outer-layer sheath | 4 outer-layer sheath control handle |
| 5 control guide wire switch | 6 control guide wire |
| 7 main body coil | 8 stent main body |
| 9 branch safety buckle | 10 branch sheath |
| 11 stent branch | 12 hot-melt part |
| 13 inner-layer graft sleeve | 14 ejection pipe |
| 15 sleeve connecting pipe | 16 graft sleeve control handle |

### DETAILED DESCRIPTION

For a better understanding of the present invention, further descriptions are given in combination with the preferred embodiments of the present invention below.

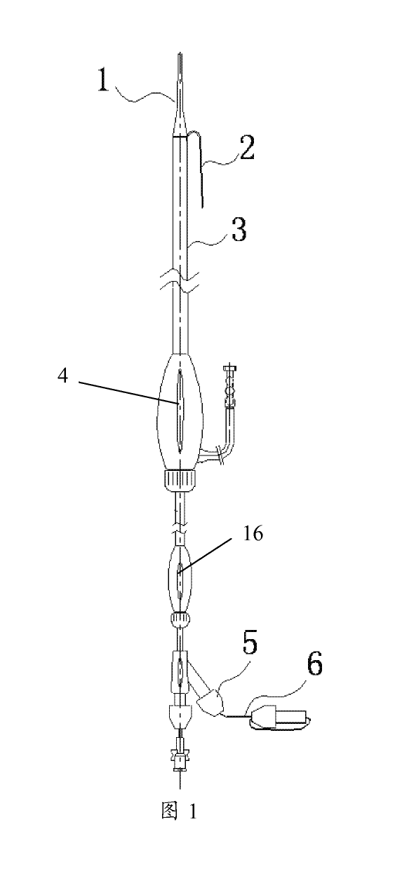

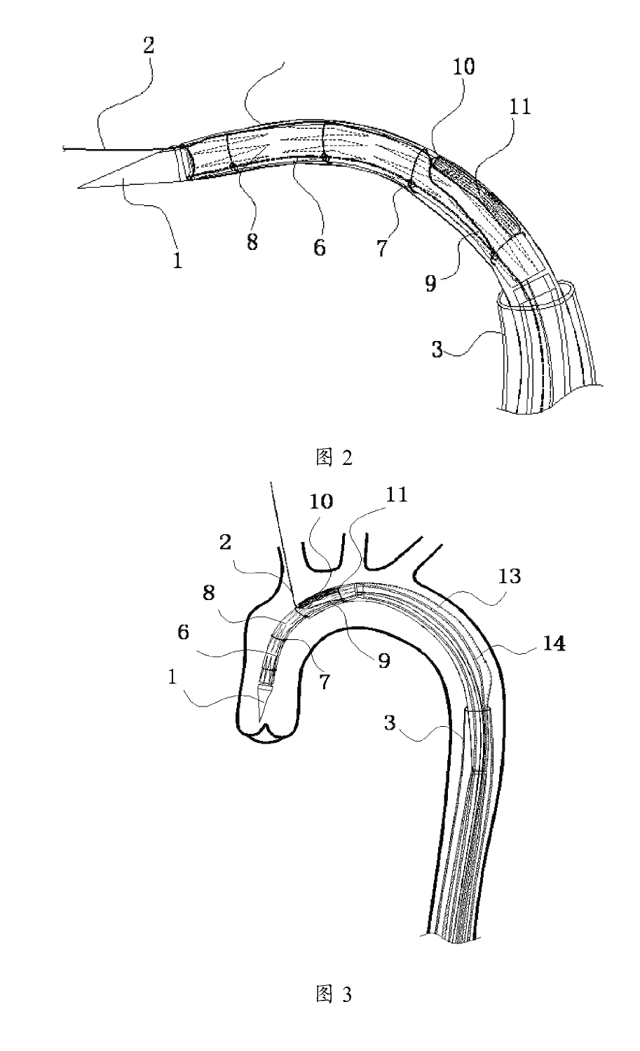

Referring to FIG. 1 and FIG. 2, a delivery system for a branched stent graft according to the present invention is provided therein with a branch guiding assembly which tows a stent branch of the stent graft, wherein the branch guiding assembly is similar to a control guiding assembly which controls a stent main body. For conciseness, only a branch guide wire 2 connected to the stent branch 11 is shown.

As can be seen from FIG. 2, a stent main body 8 of the stent graft is bound by a main body coil 7, and a control guide wire 6 which controls the towing and releasing processes of the stent main body 8 passes through respective joints of the main body coil 7, so that a detachable connection is formed between the main body coil 7 and the control guide wire 6. The stent branch 11 is bound by a branch sheath 10, and a branch guide wire 2 is connected to the branch sheath 10. In addition, the branch sheath 10 is provided thereon with a branch safety buckle 9, and similar to the main body coil 7, the branch safety buckle 9 is detachably connected to the control guide wire 6, for example, in a knotted manner. Thus, by operation of the control guide wire 6, the stent main body 8 and the stent branch 11 can be safely released simultaneously; or after the branch safety buckle 9 breaks away from the connection, an operator operates the branch sheath 10 independently on proper occasions to make the branch sheath 10 break away from the binding of the stent branch 11 and safely release the stent branch. It should be understood that the branch sheath 10 and the control guide wire 6 can be detachably connected in other similar manners, and the connective form between the branch safety buckle 9 and the control guide wire 6 can be changed correspondingly according to actual requirements. Furthermore, the delivery system shown in FIG. 1 is further provided with a graft sleeve control handle 16 in addition to a handle 4 for operating an outer-layer sheath 3.

Under an initial state, after being bound, the stent branch 11 and the stent main body 8 are entirely positioned within a double-layer sheath composed of an outer-layer sheath 3 and an inner-layer graft sleeve 13, and then delivered to the artery. In the state shown in FIG. 2, when the inner-layer graft sleeve 13 that encapsulates the stent graft has been moved out of the outer-layer sheath 3, the operator can easily deliver the inner-layer graft sleeve 13 made of a flexible polymer material through a tortuous artery by operating the delivery system, without damaging the artery.

When the stent graft has been delivered to surroundings of the location of the lesion inside a blood vessel, the stent graft connected with an ejection pipe 14 can be further moved out of the inner-layer graft sleeve 13 by operating the inner-layer graft sleeve 13, as shown in FIG. 3. In this case, a conical head 1 drives the control guide wire 6 to continue to perform towing inside the aorta blood vessel, and the branch guide wire 2 is towed into the branch blood vessel by a corresponding control mechanism (not shown).

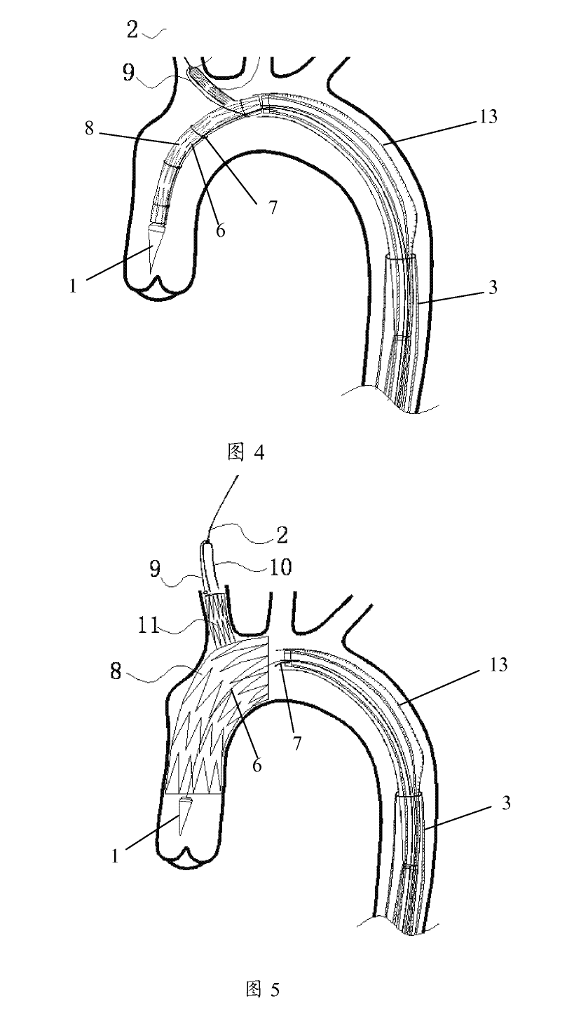

When the stent main body 8 and the stent branch 11 of the stent graft are both moved out of the inner-layer graft sleeve 13, as shown in FIG. 4, the branch guide wire 2 is operated to separate the stent branch 11 from the stent main body 8. Under such a circumstance, the stent graft is in a pre-released state, and since the stent branch 11 is still being bound in the branch sheath 10 and has not been released, and meanwhile the stent main body 8 is also being in a bound state and has not been released, the operator can control the form of the stent main body 8 and adjust the position of the stent branch 11 by means of the control guide wire 6 and the branch guide wire 2 according to the actual condition of the operation to thereby achieve an accurate positioning of the whole of the stent graft.

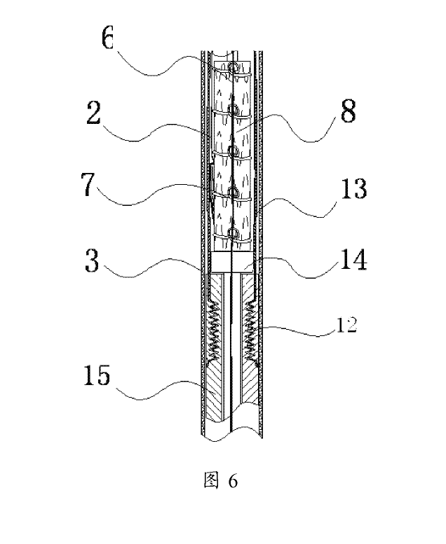

Once the positioning of the stent graft is completed, the stent graft can be safely released. Since the main body coil 7 and the branch safety buckle 9 are both detachably connected to the control guide wire 6 in a knotted manner, after the accurate positioning of the stent graft, a control guide wire switch 5 in the delivery system as shown in FIG. 1 is unscrewed, the control guide wire 6 is drawn backwards, the main body coil 7 and the branch safety buckle 9 are loosened at the joints, then the stent main body 8 starts to be released, and the branch sheath 10 of the stent branch 11 loses the fixing force at the same time to break away from the stent branch 11 under the driving of the branch guide wire 2, thereby the stent branch 11 gets rid of the binding and starts to be released, as shown in FIG. 5. During the above releasing process, the operator can also control the releasing process of the stent graft according to actual applications. After the completion of the release, the control mechanism for towing the guide wire, e.g., a guide wire head 1 of the control guide wire 6 can be removed through the stent graft that has been released, thereby the whole delivery system can be removed from the artery subsequently.

FIG. 6 further shows a connective mode for fixing the inner-layer graft sleeve 13 to another polymer pipe, e.g., a sleeve connecting pipe 15, in the delivery system of the present invention. Here, a multi-layer sleeve thermal connection technique is employed. The process of this technique is to hot-melt an intermediate-layer sleeve, e.g., the inner-layer graft sleeve 13, of one of two layers of polymer materials, e.g., PET, PTFE, etc., to make the intermediate-layer sleeve be connected and joined with the other sleeve connecting pipe 15 to thereby stably connect the inner-layer graft sleeve assembly in the delivery system. In FIG. 6, the reference sign 12 denotes the part which is hot-melt.

The above contents are descriptions of the preferred embodiments of the present invention. It should be understood that the present invention is not limited to the above specific embodiments, and those skilled in the art can make various transformations or amendments within the scope of the attached claims.

1. A delivery system for a branched stent graft, comprising a double-layer sheath containing the stent graft and a towing and releasing mechanism which tows and releases the stent graft from the sheath, wherein:
after being bound, a stent main body and a stent branch of the stent graft are movably positioned within the double-layer sheath;
the towing and releasing mechanism includes a control guiding assembly and a branch guiding assembly;
the control guiding assembly is detachably connected with the stent main body, and tows and releases the stent main body; and
the branch guiding assembly is detachably connected with the stent branch and the control guiding assembly respectively, so that the stent branch is towed by the branch guiding assembly and the stent branch is released by the control guiding assembly.

2. The delivery system for a branched stent graft according to claim 1, characterized in that:
the double-layer sheath comprises an outer-layer sheath and an inner-layer graft sleeve, wherein after movably encapsulating the stent branch and the stent main body in a complete or semi-complete manner, the graft sleeve is positioned in the outer-layer sheath.

3. The delivery system for a branched stent graft according to claim 2, characterized in that:
before being positioned in the double-layer sheath, the stent branch is enclosed in a branch sheath and is bound by the branch sheath, so that the stent branch and the stent main body are isolated from each other.

4. The delivery system for a branched stent graft according to claim 3, characterized in that:
the control guiding assembly comprises a control guide wire and a corresponding control mechanism, and a main body coil detachably binds the stent main body and is connected to the control guide wire; and
the branch guiding assembly comprises a branch guide wire and a corresponding control mechanism, the branch sheath is detachably connected to the control guide wire, and the branch guide wire connects and tows the branch sheath.

5. The delivery system for a branched stent graft according to claim 4, characterized in that:
the branch sheath is detachably connected to the control guide wire via a branch safety buckle on the branch sheath.

6. The delivery system for a branched stent graft according to claim 5, characterized in that:
the graft sleeve is made of a flexible polymer material.

7. The delivery system for a branched stent graft according to claim 6, characterized in that:
the flexible polymer material is PET or PTFE.

8. The delivery system for a branched stent graft according to claim 5, characterized in that:
the outer-layer sheath is made of a polymer material.

9. The delivery system for a branched stent graft according to claim 8, characterized in that:
the polymer material is HDPE or Pebax.

10. A method for delivering a branched stent graft to inside of a blood vessel using the delivery system for a branched stent graft according to any one of claim 1, comprising:
a) a towing step for towing the stent graft to inside of an artery by the control guiding assembly, and moving the stent graft out of the double-layer sheath;
b) a positioning step for respectively adjusting positions of the stent main body and the stent branch by the control guiding assembly and the branch guiding assembly according to a location of a lesion; and
c) a releasing step for making the control guiding assembly loosen binding of the stent graft to thereby release the stent graft.

11. The method according to claim 10, characterized in that:
in the step a), the inner-layer sheath encapsulating the stent graft is moved out of the outer-layer sheath, and then the stent graft is moved out of the inner-layer sheath.

12. The method according to claim 11, characterized in that:
a pre-releasing step b′) is further included between the steps b) and c), wherein in the step b′), the stent branch is still being bound, and the branch guiding assembly tows the stent branch to make the stent branch be separated from the stent main body and enter a branch blood vessel.
